(19) United States
(12) Patent Application Publication
(10) Pub. No.: US 2016/0081845 A1
(43) Pub. Date: Mar. 24, 2016

(54) ANTI-SNORE NASAL MODULATOR

(71) Applicant: PurrZone, Inc., Sarasota, FL (US)

(72) Inventor: Lisa Fitzgerald, Sarasota, FL (US)

(21) Appl. No.: 14/851,390

(22) Filed: Sep. 11, 2015

Related U.S. Application Data
(60) Provisional application No. 62/052,557, filed on Sep. 19, 2014.

(51) Int. Cl. A61F 5/56

(52) U.S. Cl. CPC A61F 5/56

(57) ABSTRACT

Disclosed is a nasal modulator that reduces or eliminates snoring. The disclosed nasal modulator is placed in the nasal passages and modulates air in an approximate frequency range of snoring. This modulation of inhaled air disrupts the vibration of respiratory structures by being out of phase with the normal resonance of these structures. The resonant air pressure wave is also out of phase with the inhaled modulated air. This cancellation of pressure waves and vibration of structures inhibits the loud auditory result associated with snoring.

### CROSS-REFERENCE TO RELATED APPLICATIONS

This U.S. Non-Provisional Patent Application claims priority from U.S. Provisional Patent Application No. 62/052,557 filed on Sep. 19, 2014, which is incorporated herein in its entirety.

### TECHNICAL FIELD

The present invention generally relates to the field of respiratory care, and more particularly to devices that reduce or prevent snoring.

### BACKGROUND

Snoring is the vibration of respiratory structures that results from a temporarily obstructed airway while breathing. For example, in most instances, snoring occurs while individuals sleep or begin to doze off. In certain instances snoring may be soft and unobtrusive. However, in other instances, snoring can be loud, unpleasant, and rather obtrusive for those nearby.

In addition to the unpleasant sounds that may occur during snoring, snoring may also be a sign, or first alarm, of obstructive sleep apnea (OSA). To date, the only effective treatments to reduce or prevent snoring involve surgical procedures that may, for example, ablate certain respiratory structures associated with snoring. However, the procedures are costly, invasive, and involve moderate to prolonged recovery time.

### SUMMARY

Therefore, a need exists to effectively treat and/or reduce snoring in a cost-effective manner. Disclosed is an anti-snore nasal modulator device that effectively treats and/or reduces snoring in a cost-effective manner. The disclosed anti-snore nasal modulator device includes an air-flow tube body configured for insertion into each nostril of a user, wherein the nasal air-flow tube body comprises a vibrating vane positioned co-axially relative to nasal air flow such that the vibrating vane is configured to modulate nasal air flow during inhalation such that the modulating vane is configured to suppress or destroy nasal laminar flow air flow to reduce or prevent snoring.

In certain aspects, the anti-snore nasal modulator device includes an air-flow tube body configured for insertion into each nostril of a user, wherein the nasal air-flow tube body includes a vibrating vane positioned co-axially relative to nasal air flow such that the vibrating vane is configured to modulate nasal air flow during inhalation such that the modulating vane is configured to suppress or destroy nasal laminar flow air flow to reduce or prevent snoring.

In certain aspects, the air-flow tube body includes two elongate cylindrical bodies, each elongate cylindrical body having an elastically, radially deformable inlet adapted for insertion into the nostril of the user and a non-deformable, rigid outlet that is in fluid communication with the elastically, radially deformable inlet.

In certain aspects, two elongate cylindrical bodies have a parallel axial orientation relative to each other about a longitudinal axis of each elongate cylindrical body.

In certain aspects, the two elongate cylindrical bodies are joined together at a portion of the device in closer proximity to the non-deformable, rigid outlet than the elastically, radially deformable inlet.

In certain aspects, the anti-snore nasal modulator device further includes two retaining loops positioned on an outer portion of the device where the two elongate cylindrical bodies are joined together, the retaining loops forming an opening for receiving a retaining member adapted to secure the device to the user's nostrils, each opening having a longitudinal axis extending parallel relative to the longitudinal axis of each elongate cylindrical body.

In certain aspects, the vibrating vane is positioned within each elongate cylindrical body.

In certain aspects, inner diameter of the anti-snore nasal modulator is adapted to receive the vibrating vane by a sliding engagement.

In certain aspects, the vibrating vane engages the inner diameter of the anti-snore nasal modulator about the elastically, radially deformable inlet and extends in a direction toward the non-deformable, rigid outlet.

In certain aspects, the anti-snore nasal modulator includes two elongate cylindrical bodies, each elongate cylindrical body having a disposable elastically, radially deformable inlet adapted for insertion into the nostril of the user and a non-deformable rigid outlet, wherein an inner diameter of the disposable elastically, radially deformable inlet is adapted to fit over and engage via a friction fit to an outer diameter of the non-deformable rigid outlet; the disposable elastically, radially deformable inlet is in fluid communication with the elastically, radially deformable inlet; and the vibrating vane engages via a sliding engagement to an inner diameter of the anti-snore nasal modulator about the elastically, radially deformable inlet and extends in a direction toward the non-deformable, rigid outlet. In certain aspects, an end portion of the vibrating vane is positioned over an inner diameter of the non-deformable, rigid outlet and extends perpendicularly relative to a longitudinal axis of the elongate cylindrical body such that the vibrating vane is adapted for translational movement between opposite sides of an inner diameter of the device above the inner diameter of the non-deformable, rigid outlet while in use thereby ablating audible snoring sounds.

Additional features, aspects and advantages of the invention will be set forth in the detailed description which follows, and in part will be readily apparent to those skilled in the art from that description or recognized by practicing the invention as described herein. It is to be understood that both the foregoing general description and the following detailed description present various embodiments of the invention, and are intended to provide an overview or framework for understanding the nature and character of the invention as it is claimed. The accompanying drawings are included to provide a further understanding of the invention, and are incorporated in and constitute a part of this specification.

### BRIEF DESCRIPTION OF THE DRAWINGS

These and other features, aspects and advantages of the present invention are better understood when the following detailed description of the invention is read with reference to the accompanying drawings, in which:

FIG. 1 is a perspective view of the anti-snore nasal modulator;

FIG. 2 is a top plan view of the anti-snore nasal modulator;

FIG. 3 is a front plan view of the anti-snore nasal modulator;

FIG. 4 is a side plan view of the anti-snore nasal modulator;

FIG. 5 is a bottom plan view of the anti-snore nasal modulator;

FIG. 6 is a simplified view of FIG. 3;

FIG. 7 is a section view in the direction of arrows 7-7 in FIG. 6;

FIG. 8 is a simplified view of FIG. 4;

FIG. 9 is a section view in the direction of arrows 9-9 in FIG. 8; and

FIG. 10 is an exploded view of the anti-snore nasal modulator.

FIG. 11 is a section view of the nose and mouth

### DETAILED DESCRIPTION

The present invention will now be described more fully hereinafter with reference to the accompanying drawings in which exemplary embodiments of the invention are shown. However, the invention may be embodied in many different forms and should not be construed as limited to the representative embodiments set forth herein. The exemplary embodiments are provided so that this disclosure will be both thorough and complete, and will fully convey the scope of the invention and enable one of ordinary skill in the art to make, use and practice the invention. Like reference numbers refer to like elements throughout the various drawings.

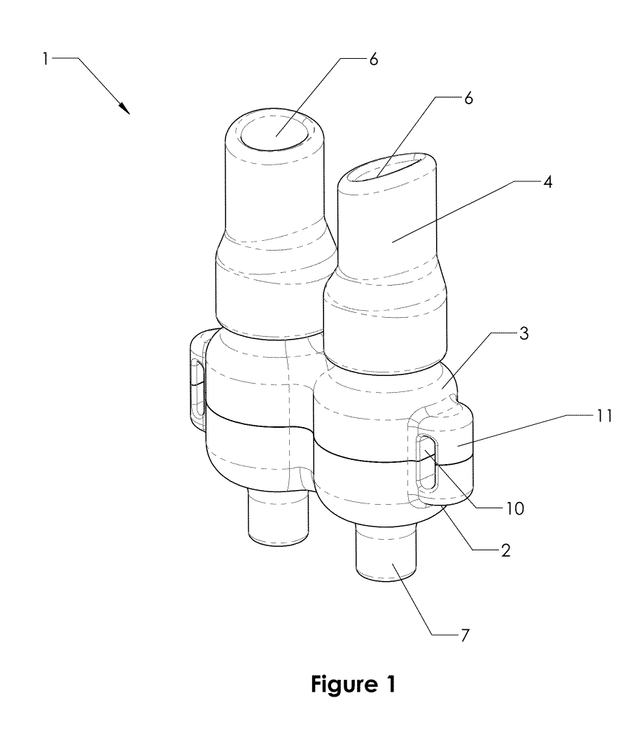

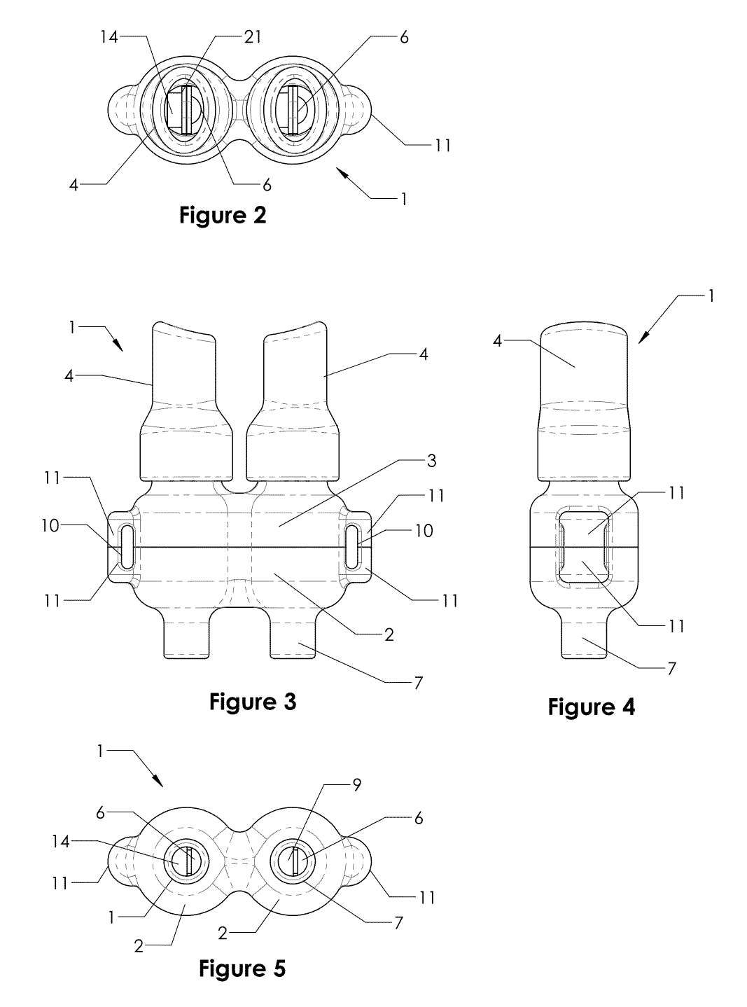

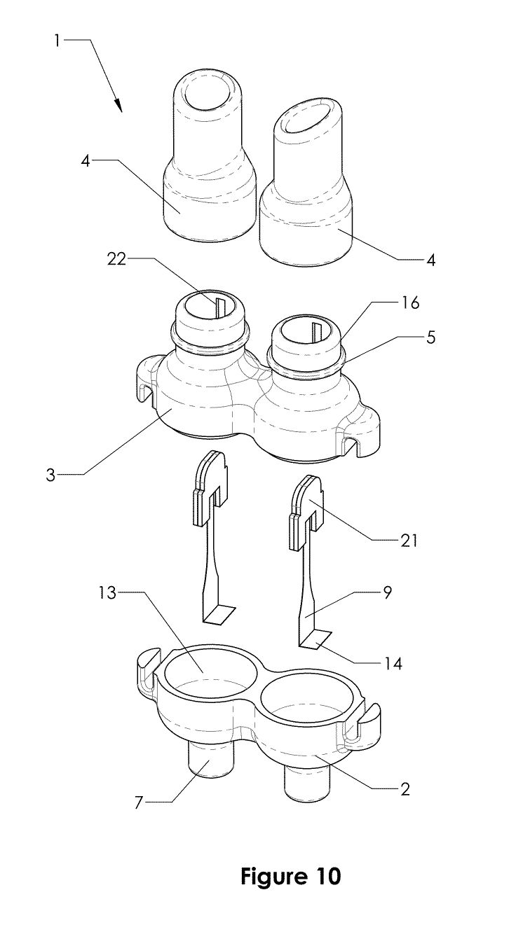

Referring now to the drawings, and first to FIGS. 1, 2, 3, 4, 5, and 10, the nasal modulator 1 includes a lower housing 2, an upper housing 3, two nasal inserts 4 and 4A, and two modulator vanes 9. A strap aperture 10 in strap retainer boss 11 provides strap mounting for a strap (not shown). This anchor point located on each side of the housing is used to attach the nasal modulator 1 to the head. The lower housing 2 and upper housing 3 are made from an injection-molded plastic.

The housings and nasal inserts form two air-flow tubes 6 and 6A from the inlet ports 7 and 7A to the outlet ports 16 and 16A on each side of the housing. Two air paths are required, one for each nostril, due to alternating turgescence of the inferior turbinates of the nasal passages normal in 80% of the population. This turgescence causes a blockage of air flow through the nasal cavity alternating approximately every 4 hours for a normal human.

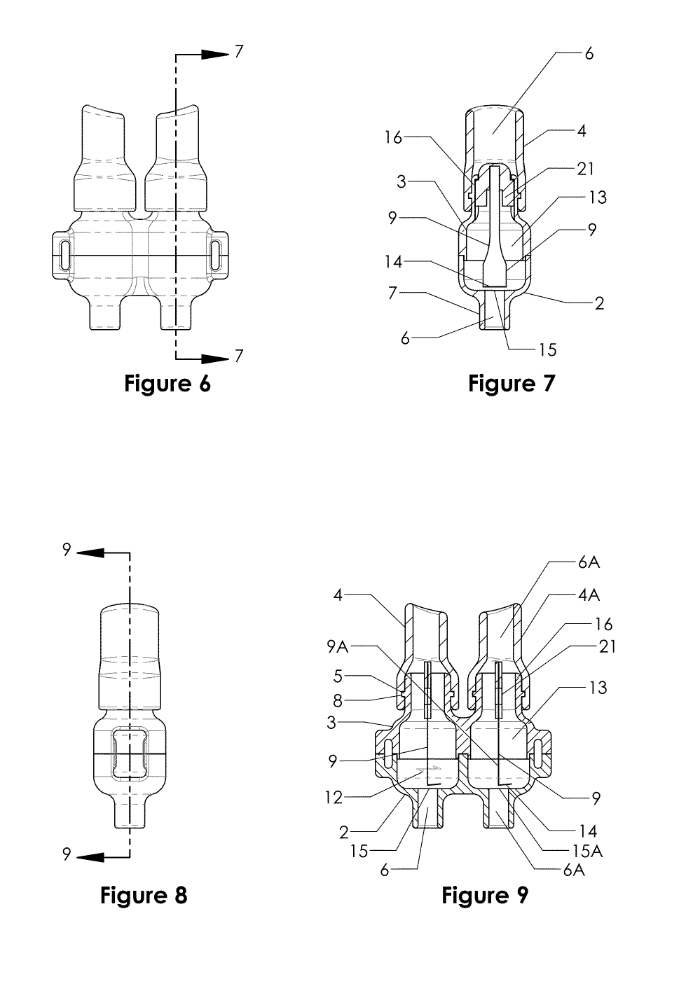

Referring to FIGS. 6, 7, 8, 9, and 10, a modulator vane 9 is mounted to the upper housing 3 by means of vane mount 21, captured by vane retainer slot 22 in upper housing 3. The modulator vane 9 is made of stainless steel. The inhaled air flows from the inlet port 7 through the inlet aperture 15 in cavity 13 and across the modulator vane 9. The vane deflector 14 causes unequal air distribution between modulator vane 9 and the opposite side 9A. This pressure differential vibrates modulator vane 9, resulting in vane displacement 12. Vane displacement 12 moves vane deflector 14 back and forth across the inlet aperture 15 to modulate the inlet 7 airflow in cavity 13. This airflow modulation is translated through the air-flow tubes 6 and 6A to each nostril.

The nasal inserts 4 and 4A are pushed onto the outlet port 16 and held in place by means of the elasticity of nasal insert 4 to stretch over retainer ring 5 on outlet port 16 and lock into retainer groove 8. The nasal inserts are removable and are manufactured in various sizes to accommodate anatomy differences of the nostril. For comfort, the two nasal inserts 4 and 4A are made from a soft silicone or urethane rubber.

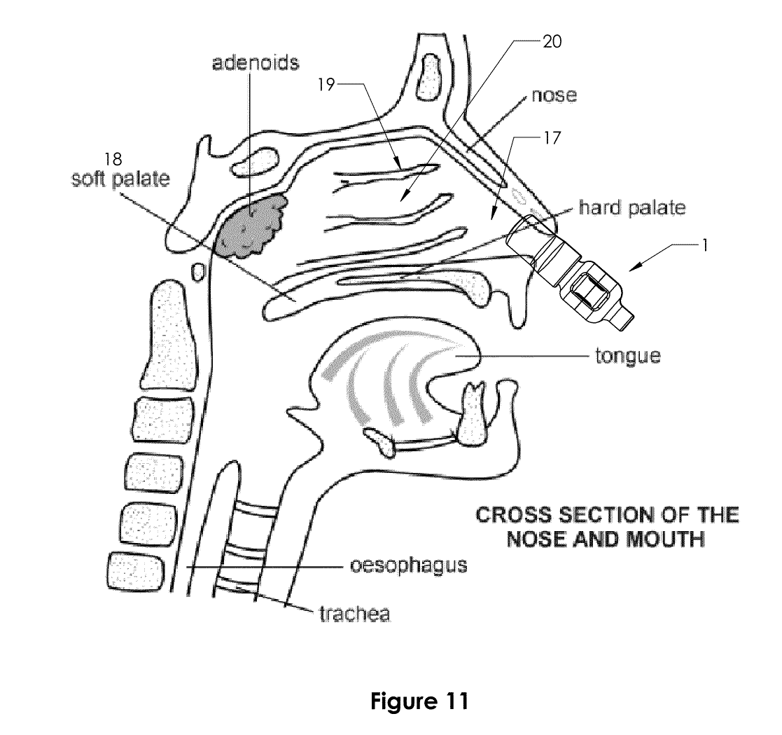

Referring to FIG. 11, nasal modulator 1 is placed in each nostril 17 by means of the two nasal inserts 4 and 4A. The modulated air flow from nasal modulator 1 enters nasal cavity 20 and prevents vibration of nasal structures 19 and soft palate 18. This is done by replacing normal laminar air flow with modulated air (pulsed air). This modulated air also cancels out any resonant audio frequency generated by the internal dimensions of nasal cavity 20. The modulated pressure pulse from nasal modulator 1 is out-of-phase with the snore-generated audio pressure wave. This quiets or eliminates the snore audio report.

The foregoing description provides embodiments of the invention by way of example only. It is envisioned that other embodiments may perform similar functions and/or achieve similar results. Any and all such equivalent embodiments and examples are within the scope of the present invention and are intended to be covered by the appended claims.

The invention claimed is:

1. An anti-snore nasal modulator device comprising:
an air-flow tube body configured for insertion into each nostril of a user, wherein:
the air-flow tube body comprises a vibrating vane positioned co-axially relative to nasal air flow such that the vibrating vane is configured to modulate nasal air flow during inhalation such that the modulating vane is configured to suppress or destroy nasal laminar flow air flow to reduce or prevent snoring.

2. The anti-snore nasal modulator device of claim 1, wherein the air-flow tube body comprises:
two elongate cylindrical bodies, each elongate cylindrical body having an elastically, radially deformable inlet adapted for insertion into the nostril of the user and a non-deformable, rigid outlet that is in fluid communication with the elastically, radially deformable inlet.

3. The anti-snore nasal modulator device of claim 3, wherein the two elongate cylindrical bodies have a parallel axial orientation relative to each other about a longitudinal axis of each elongate cylindrical body.

4. The anti-snore nasal modulator device of claim 4, wherein the two elongate cylindrical bodies are joined together.

5. The anti-snore nasal modulator device of claim 4, further comprising two retaining loops positioned on an outer portion of the device, the retaining loops forming an opening for receiving a retaining member adapted to secure the device to the user's nostrils, each opening having a longitudinal axis extending parallel relative to the longitudinal axis of each elongate cylindrical body.

6. The anti-snore nasal modulator device of claim 5, wherein the vibrating vane is positioned within each elongate cylindrical body.

7. The anti-snore nasal modulator device of claim 6, wherein an inner diameter of the anti-snore nasal modulator is adapted to receive the vibrating vane by a sliding engagement.

8. The anti-snore nasal modulator device of claim 7, wherein the vibrating vane engages the inner diameter of the anti-snore nasal modulator about the elastically, radially deformable inlet and extends in a direction towards the non-deformable, rigid outlet.

9. The anti-snore nasal modulator device of claim 8, wherein an end portion of the vibrating vane is positioned over an inner diameter of the non-deformable, rigid outlet and extends perpendicularly relative to the longitudinal axis of the elongate cylindrical body such that the vibrating vane is adapted for translational movement between opposite sides of an inner diameter of the device immediately above the inner diameter of the non-deformable, rigid outlet while in use thereby ablating audible snoring sounds.

10. The anti-snore nasal modulator of claim 1, wherein the anti-snore nasal modulator comprises two elongate cylindrical bodies joined together, each elongate cylindrical body having a disposable elastically, radially deformable inlet adapted for insertion into the nostril of the user and a non-deformable rigid outlet, wherein:
an inner diameter of the disposable elastically, radially deformable inlet is adapted to removably fit over and engage via a friction fit an outer diameter of the non-deformable rigid outlet;
the disposable elastically, radially deformable inlet is in fluid communication with the elastically, radially deformable inlet; and
the vibrating vane is secured in each elongate cylindrical body via a sliding engagement to an inner diameter of the anti-snore nasal modulator and extends in a direction towards the non-deformable, rigid outlet.

11. The anti-snore nasal modulator of claim 10, wherein an end portion of the vibrating vane is positioned immediately over an inner diameter of the non-deformable, rigid outlet and extends perpendicularly relative to a longitudinal axis of the elongate cylindrical body such that the vibrating vane is adapted for translational movement between opposite sides of an inner diameter of the device above the inner diameter of the non-deformable, rigid outlet while in use thereby ablating audible snoring sounds.
